SEQUENCE LISTING (6 sequences)

SEQ ID NO: 1 - DNA, 20 bp, artificial
  synthetic primer
gccttcctaccccaccatgt

SEQ ID NO: 2 - DNA, 19 bp, artificial
  synthetic primer
cctgaatcgcgcatcgtta

SEQ ID NO: 3 - DNA, 21 bp, artificial
  synthetic primer
tacgagacgagaaaaagacag

SEQ ID NO: 4 - DNA, 19 bp, artificial
  synthetic primer
gtcctaatcgggtaaaaga

SEQ ID NO: 5 - DNA, 17 bp, artificial
  synthetic primer
cccctaaggtcaaagaa

SEQ ID NO: 6 - DNA, 16 bp, artificial
  synthetic primer
ggtaggcttggcactc

(19) United States
(12) Patent Application Publication
(10) Pub. No.: US 2023/0183821 A1
(43) Pub. Date: Jun. 15, 2023

(54) FIELD METHOD FOR DETECTING THE PRESENCE AND MULTI-RESISTANCE OF A PEST, IN PARTICULAR ZYMOSEPTORIA TRITICI, IN CEREAL CROPS

(71) Applicant: Anova-Plus, Evry (FR)

(72) Inventors: Marc Masson, Ris-Orangis (FR); Natacha Testas, L'Haÿ-les-Roses (FR)

(21) Appl. No.: 17/999,326

(22) PCT Filed: May 17, 2021
(86) PCT No.: PCT/FR2021/050852

§ 371 Date: Dec. 29, 2022

(30) Foreign Application Priority Data
May 20, 2020 (FR) FR2005331

(51) Int. Cl. C12Q 1/6895

(52) U.S. Cl. CPC C12Q 1/6895

(57) ABSTRACT

The present disclosure relates to the field of diagnostics for monitoring strains or breeds of pests (fungi, bacteria, viruses and insects). More specifically, it relates to a method for detecting and characterizing, directly in the field, the presence of a strain/breed of pest and its resistance type characteristics. It also relates to a ready-to-use field diagnostic kit, referred to as a “Pedestrian Alert,” for implementing the method. It is used, in particular, for detecting and characterizing the strains of the fungus Zymoseptoria tritici, which is responsible for Septoria in wheat, which can destroy more than 30% of the harvests of straw cereals such as soft wheat, barley and durum wheat.

### CROSS-REFERENCE TO RELATED APPLICATIONS

This application is a national phase entry under 35 U.S.C. § 371 of International Patent Application PCT/FR2021/050852, filed May 17, 2021, designating the United States of America and published as International Patent Publication WO 2021/234260 A1 on Nov. 25, 2021, which claims the benefit under Article 8 of the Patent Cooperation Treaty to French Patent Application Serial No. FR2005331, filed May 20, 2020.

### TECHNICAL FIELD

The present disclosure relates to the field of diagnostics for monitoring strains or breeds of pests (fungi, bacteria, viruses and insects). More specifically, it relates to a method for detecting and characterizing, directly in the field, the presence of a strain/breed of a pest and its resistance type characteristics, TSR and MDR, to pesticides (modes of action), its virulence characteristics compared to host plants, etc.

It also concerns a ready-to-use field diagnostic kit, referred to as “Pedestrian-Alert,” for implementing the method. It is used, in particular, for detecting and characterizing strains of the fungus Zymoseptoria tritici, which is responsible for Septoria in wheat, which can destroy more than 30% of the harvests of straw cereals such as durum wheat, soft wheat and barley.

### BACKGROUND

The present disclosure relates to the field of field diagnostics used for detecting and characterizing strains of pests concerning their resistance to pesticides. In particular, it concerns the characterization of the wheat fungus Zymoseptoria tritici. This pathogen is responsible for the disease known as “Septoria.” This disease, which mainly affects wheat, is found in all wheat-growing areas around the world. It is indeed one of the most detrimental foliar diseases of these crops, leading to substantial yield losses (up to 40 quintals/ha). Septoria is the leading foliar disease of wheat in France; it is present every year. It takes the form of so-called “septorian” spots and can lead to a yield loss of around 30 to 50%.

Septoria spreads during rains or ambient humidity. The pycnidia, haploids resulting from mitosis, fill up with water and swell. The pycnidia then expel spores, also haploids, in the form of a transparent sporiferous jelly called “cirrus.” The spores are then disseminated by splashing rain. Septoria can be difficult to detect, in particular, because the symptomatic spots of the disease can be equated to simple physiological spots without progression. Thus, the rapid and reliable determination of the presence of the pathogen is essential to allow effective treatment of cereals.

To combat this infection, antifungals from the QoI (Quinone outside Inhibitors) family, DMIs (Demethylase Inhibitors) more commonly called Triazoles, or SDHIs (Succinate DesHydrogenase Inhibitors) are generally used.

However, due to the single-site mode of action of commonly used antifungals and the pressure induced by the massive use of these fungicides, resistance has appeared in Zymoseptoria tritici, namely target-site resistance (TSR).

Other types of resistance, referred to as non-target-site resistance, have also appeared, bringing independent genetic mechanisms into play; they are grouped together under the name of “multi-drug resistance” or MDR and affect all the modes of action of the fungicides used, drastically reducing their effectiveness. MDR also affects the effectiveness of many treatments in the clinical sector (cancer, antibiotics, antifungals) as well as in agriculture. In the case of phytopathogenic fungi, it reduces the effectiveness of several fungicidal modes of action, requiring the use of higher doses. MDR is caused by the overexpression of membrane transporters that expel toxic molecules from cells. In the fungus responsible for Septoria in wheat, Zymoseptoria tritici, three types of inserts in the MFS1 (Major Facilitator Superfamily gene 1) gene promoter encoding a transporter of the MFS family are at the origin of MDR phenotypes in isolates of the Septoria fungus.

This phenomenon is particularly worrying in the current context aimed at reducing inputs, including pesticides (e.g., EcoPhyto II plan). With an average frequency of 26% of multidrug-resistant isolates in French populations of Z. tritici in 2019 (A.-S. Walker, com. pers.), the application of reduced doses of fungicides may not only prove ineffective, but also promote survival and therefore select multi-resistant isolates. The development of tools making it possible to study the evolution of the frequencies of TSR- and MDR-type resistances in the field based on the treatments applied is essential.

In this context, it is essential to quickly diagnose plants affected by Septoria linked to TSR and/or MDR resistance.

The state of the art teaches many detection methods using the quantitative PCR method. However, the reliability of these methods can be uncertain depending on the detection method used. Indeed, when carrying out a conventional PCR method, polymers can form, resulting from a mismatch of the primer sequences and the targeted DNA sequences. The formation of these polymers, in particular, of dimers, plays a determining role in the reliability of the results obtained during the indication of the amplified sequences. The presence of polymerizations indeed leads to false positives, especially if the indication is done on an immunological strip. Neutralizing these polymers is therefore essential to obtain reliable results.

To limit the formation of these polymers, the state of the art offers various solutions.

Document WO2006112818A2 describes a method reducing the formation of dimers by adding modified nucleobases to the 3′ end of the primers. The document provides for polynucleotides comprising at least one modified pyrimidine nucleobase and no more than 4 nucleotides at the 3′ end of the polynucleotide.

Document EP2814976 B1 proposes a method for reducing the nonspecific formation of dimeric or polymeric nucleic acid in which 2-amino-deoxyadenosine (2-amino-dA), 2-thio-deoxythymidine (2-thio-dT) or other nucleotide analogs of interest are incorporated into the random hexamers used for the amplification of the target nucleic acid.

Although functional, the methods known from the state of the art are imperfect. Indeed, they propose the design of specific Single Nucleotide Polymorphism (SNP) primers via a PCR method with additions of nucleobases, which is more complex, requires more technical expertise and requires more time.

One of the detection methods already known in the state of the art is the method using recombinase polymerase amplification, which makes it possible to highlight the presence of a specific marker characterizing a pest (also known as Flashdiag). This method is characterized by: sampling a piece of leaf showing a symptom, extracting DNA or RNA from the sample by grinding and filtration steps, then placing the sample in a tube containing the probes and primers necessary for detection and subjecting the mixture to recombinase polymerase amplification, then finally, placing the tube in a U-star type cassette for the indication of the result owing to specific DNA probes.

There is a real need to have a field diagnostic tool that can quickly detect the presence of a pest and identify, for example, its resistance to pesticides to guide the choice of treatment(s) to apply.

### BRIEF SUMMARY

The present disclosure relates to a field method, that is to say, outside the laboratory, directly near the field, for detecting the presence of a pest. This method also makes it possible to determine the resistance of pests to pesticide treatments, more particularly to detect TSR and/or MDR resistance, to monitor its progression in the field. More particularly, the present disclosure relates to the detection of an infection of straw cereals (barley, durum wheat, soft wheat) by the pathogenic agent Zymoseptoria tritici.

The method according to the present disclosure comprises collecting a leaf sample of the plant suspected of being infected, or insect vectors of parasites or adventitious plants (weeds) present in the crop, extracting the DNA or RNA of the sample, using PCR or RT-PCR to amplify DNA or RNA sequences of the pest to determine i) the presence of the latter owing to a pair of specific primers making it possible to amplify a sequence characteristic of the pest, ii) the possible presence of at least one mutation responsible for resistance to a phytopharmaceutical treatment, this amplification being carried out in the presence of interfering DNA complementary to the parts of the primers capable of forming polymers between them in order to avoid the formation of aspecific bands on the immunological strips responsible for false positives; the presence of the pest and possibly of a resistance of the latter is indicated on immunological strips (for example, of the DAS-Elisa nitrocellulose type).

The present disclosure also relates to a kit for detecting the presence of a pest and its resistance to pesticide treatments, suitable for use in the field, in particular, for detecting strains of the pathogen Zymoseptoria tritici and their resistance to antifungal treatments.

The present disclosure meets the need of the agricultural world by proposing a rapid detection method making it possible to highlight both the presence of a pest and the multi-resistance of the latter to phytopharmaceutical treatments owing to quantitative PCR amplification and detection on immunological strips. More particularly, this method applies to the detection of the pathogen Zymoseptoria tritici.

PCR amplification allows rapid and effective determination of a pest and a mutation, in particular, the mutation inducing multi-resistance. The DNA or RNA extract of the sample is directly introduced into a mixture containing the specific primers of the pest and others specific to the mutations responsible for the resistances, and the whole is subjected to a PCR amplification step followed by the indication of the results owing to an immunological strip. The results are obtained by detection via antibodies bound to the strip of an antigen marker bound to the end of the amplified primers. This method does not require any DNA or RNA probe and allows a simple and rapid sequence of steps.

During a PCR, the primers used in the mixture can form polymers. As a general rule and in reading on gel, this only causes a loss of effectiveness of the system, which is more or less significant depending on the polymer formed. Conversely, when the technology uses indication on an immunological strip, this also causes the formation of “false positives.” Indeed, the strip is able to identify a polymer if it has the two necessary antigen markers at each end of the dimer or polymer formed. In this case, a band will light up while the sample is negative.

Thus, to avoid the appearance of false positives and increase the reliability of the method, it has been demonstrated, unexpectedly, that the addition of interfering DNA acts significantly on the formation of polymers between primers, thus avoiding the appearance of aspecific bands responsible for false positives. Contrary to what the prior art teaches, the added interfering DNA is not linked here to a primer. The method is therefore robust and reliable.

Owing to the use of interfering DNA, it is possible to increase the number of primers present in the mixtures while being able to avoid the appearance of false positives. Thus, the present disclosure proposes to simultaneously detect 4 markers in the same PCR reaction by using two indicating bands for 2 markers each. This technology therefore provides access to a powerful field diagnostic tool for appropriate decision-making.

The kit according to the present disclosure is easy and quick to use, the result being available in less than one hour. In addition, the kit has been designed to allow use by people without any laboratory experience, without special protective equipment and without risk to the environment or the handler (without toxic component). The result is presented in strip form and is easy to interpret (point of care quick diagnosis).

### BRIEF DESCRIPTION OF THE DRAWINGS

FIG. 1: Schematic representation of the steps of the method for detecting a pest according to the present invention.

FIG. 2: General principle of indication on immunological strip.

### DETAILED DESCRIPTION

A first object of the present disclosure relates to a field method for detecting the presence of a pest in a crop, comprising the steps of:
a. Collecting a leaf sample of the plant suspected of being infected, or insect vectors of parasites, or weeds present in the crop;
b. Extracting DNA or RNA from the sample;
c. Using PCR to amplify the DNA or RNA sequences of the pest in order to determine the presence of the pest owing to a pair of specific primers making it possible to amplify a sequence characteristic of the pest; and
d. Indicating the presence of the pest on immunological strip;

wherein the amplification step is carried out in the presence of interfering DNA complementary to the parts of the primers capable of forming polymers between them in order to avoid the formation of aspecific bands on the immunological strips responsible for false positives.

This method makes it possible to determine whether the plant suspected by the farmer of an attack by a pest is indeed affected or whether there is another pathology or a physiological problem of the plant involved. It also makes it possible to determine whether the pest in question has resistance to a phytopharmaceutical treatment. Owing to several pairs of primers that are specific to the pest and others specific to the main mutations responsible for one or more resistances to phytosanitary treatments, this method offers a complete diagnosis.

In a preferred embodiment of the present disclosure, this method makes it possible to detect, in addition to the presence of a first pest, the presence of at least one other pest and/or to determine at least one characteristic associated with a pest chosen from target-related pesticide resistance, multi-drug resistance, heat resistance, drought or humidity resistance.

Thus, the method makes it possible to simultaneously obtain up to 4 results associated with detecting and/or characterizing one or more pests from the same amplification reaction. In such an embodiment, the indication will be carried out using two bands that will both be impregnated in the same amplification reaction mixture.

Within the meaning of the present disclosure, the term “pest” means any biologically active agent responsible for damage to plants, such as viruses, bacteria, fungi or insects. This concept also includes weeds that “pollute” crops and reduce yields. The insect can be the pest or the vector of the pest.

The term “phytopharmaceutical treatment” or “pesticide” means any preparation intended to protect plants and crop products from the nuisance of pests by destroying them, or by using repellents in the case of insects.

The term “interfering DNA” means small DNA sequences complementary to part of a primer. When mismatched during amplification, this identified part of the primer can form polymers with the other primers. This then creates a false positive reading of the results on the strips. Owing to this total complementarity to the corresponding part of the primer, the interfering DNA sequence will compete with the other primers and prevent polymer formation by avoiding pairings between them. These sequences are short, generally less than 15 nucleotides, for example, about ten nucleotides. The concentration of these interfering DNAs and the interfering DNA/primer ratio must be adapted so as not to harm the effectiveness of the PCR.

In a preferred embodiment, the duplex format, i.e., two results (2 bands) on the same strip, the detection on the strip is done owing to:
a. The presence of antibodies bound to the strip and forming three distinct bands, each band comprising antibodies specific for a particular antigen marker;
b. the presence of a specific antigen marker at the 5′ end of one of the forward or reverse primers and the presence of an antigen marker common to all the pairs of primers at the 5′ end of the other primer, the markers being recognized by an antibody specific for one of the bands of the strip; and
c. antibodies capable of binding to the antigen marker common to all the pairs of primers and carrying an indicating system.

In a particular embodiment, the method comprises two of the three bands making it possible to indicate the presence of the pest in the sample and the existence of resistance of the latter to pesticides, respectively, and the third is a control of the functionality of the indicating system.

The antigen markers can be chosen from markers well known to those skilled in the art such as biotin, digoxigenin, FAM, etc.

In a preferred embodiment, the detection method according to the present disclosure is applied to the detection of multi-resistance (also called “Multi-Drug Resistance” or MDR) to antifungals of the pathogen Zymoseptoria tritici, responsible for Septoria, in particular, on straw cereals such as soft wheat, durum wheat and barley; the PCR amplification uses a mixture comprising the following sequences:
A pair of specific primers making it possible to detect the presence of the pest comprising sequences SEQ ID NO.1 and SEQ ID NO.2;
A pair of primers allowing the detection of multi-resistance comprising a primer of sequence SEQ ID NO.3 non-specific for multi-resistance and a primer chosen from:
a primer of sequence SEQ ID NO.4 specific for multi-resistance I;
a primer of sequence SEQ ID NO.5 specific for multi-resistance II;
a primer of sequence SEQ ID NO.6 specific for multi-resistance III;
Two interfering DNAs of sequences SEQ ID NO.7 and SEQ ID NO.8.

Multi-resistance I, II and III of the pathogen Zymoseptoria tritici is determined by the following sequences, respectively:
sequence SEQ ID NO.4;
sequence SEQ ID NO.5;
sequence SEQ ID NO.6;
each in association with the primer of sequence SEQ ID NO.3.

This method makes it possible to determine the presence of the pathogen Zymoseptoria tritici on straw cereals, such as soft wheat, barley and durum wheat, and to determine whether this pathogen has multi-resistance induced by a mutation. The interfering DNAs of DNA SEQ ID NO.7 and SEQ ID NO.8 make it possible to neutralize polymer formation, which is responsible for the loss of effectiveness of the PCR method when reading the results on gel and for the appearance of false positives when reading the results on a strip.

A second object of the present disclosure relates to a kit for detecting the presence of a pest suitable for use in the field comprising:
A lyophilized PCR reaction mixture comprising:
   a pair of specific primers making it possible to amplify a sequence characteristic of the pest
   interfering DNAs complementary to the parts of the primers capable of forming polymers between them
   a polymerase amplification enzyme
At least one immunological indicating strip making it possible to indicate the presence of the pest.

In order to provide a more complete diagnosis, in a preferred embodiment of the present disclosure the kit also comprises at least one other pair of primers making it possible either to detect the presence of another pest, or to determine at least one characteristic associated with a pest selected from target-related pesticide resistance, multi-drug resistance, heat resistance, drought or humidity resistance.

In a particular embodiment, the kit makes it possible to detect the presence of a pest and to characterize a pesticide resistance of this pest; in this kit, the PCR mixture comprises, in addition to the pair of specific primers making it possible to amplify a sequence characteristic of the pest and of the interfering DNAs, one or more pairs of specific primers making it possible to amplify at least one mutation responsible for a pesticide resistance in the pest. In this configuration, the strip comprises three distinct bands formed by the binding of antibodies specific for a particular antigen marker, namely respectively:
an antibody specific for an antigen marker located at the 5′ end of one of the primers allowing the amplification of a sequence specific for the pest;
an antibody specific for an antigen marker located at the 5′ end of one of the primers allowing the amplification of a sequence specific for a resistance; and
an antibody specific for an antigen marker common to all the pairs of primers making it possible to indicate the bands.

The kit can comprise 2 immunological strips to be used simultaneously to indicate 4 results from the same amplification. These results are chosen from detecting the presence of at least one pest and determining at least one characteristic associated with a pest chosen from target-related pesticide resistance, multi-drug resistance, heat resistance, drought or humidity resistance. This type of kit comprising 4 markers is efficient and very informative for the user while having a limited cost because these 4 results are obtained in a single operation. Using interfering DNA is particularly crucial to avoid the appearance of false positives linked to the complexity of the PCR mixture (high number of primers).

In a particularly preferred embodiment, the kit is applied to the detection of multi-drug resistance of the fungus Zymoseptoria tritici, which is responsible for Septoria, in particular, in straw cereals such as soft wheat, barley and durum wheat, in which the lyophilized PCR mixture comprises:
The primers for detecting the presence of the pathogen are of sequence SEQ ID NO.1 and SEQ ID NO.2;
The primers allowing multi-resistance detection comprise a primer of sequence SEQ. ID NO. 3 non-specific for multi-resistance and a primer chosen from:
   a primer of sequence SEQ ID NO.4 specific for multi-resistance I;
   a primer of sequence SEQ ID NO.5 specific for multi-resistance II;
   a primer of sequence SEQ ID NO.6 specific for multi-resistance III;
The interfering DNAs are of sequences SEQ ID NO.7 and SEQ ID NO.8.

The indicating strip, in duplex format, comprises three distinct bands formed by binding antibodies specific for a particular antigen marker, namely respectively:
an antibody specific for an antigen marker located at the 5′ end of one of the primers allowing the amplification of a sequence specific for Zymoseptoria tritici;
an antibody specific for an antigen marker located at the 5′ end of one of the primers allowing the amplification of a sequence specific for pesticide resistance, in particular, a multi-resistance; and
an antibody specific for an antigen marker common to all the pairs of primers making it possible to indicate the bands.

The present disclosure also relates to a field method for detecting the presence of a pest in a crop and possibly of one of its resistances of interest, using a kit as defined above, and comprising the following steps:
A) Collecting a leaf sample suspected of being infected, or insect vectors of parasites, or weeds present in the crop;
B) Extracting DNA or RNA from the sample;
C) Introducing the extracted DNA or RNA into a tube containing the lyophilized PCR mixture as defined above;
D) Performing PCR amplification (between 30 and 40 cycles); and
E) Introducing one or two immunological strips for impregnation in the tube containing the amplicons to reveal whether the pest is present and possibly whether it presents a resistance of interest.

Resistances of interest, in particular, include target-related pesticide resistance, multi-drug resistance, heat resistance, drought or humidity resistance.

In a preferred embodiment, duplex format, this method is applied to the detection of the presence of the pathogen Zymoseptoria tritici and its resistance to fungicides.

### EXAMPLES

### Example 1: Detection and Characterization of Strains of the Zymoseptoria tritici Pathogen with Indication of Two Markers

The present disclosure is illustrated with the aid of the following example and presents a particular embodiment of the present disclosure, namely the application of the method for detecting and characterizing strains of the Zymoseptoria tritici pathogen.

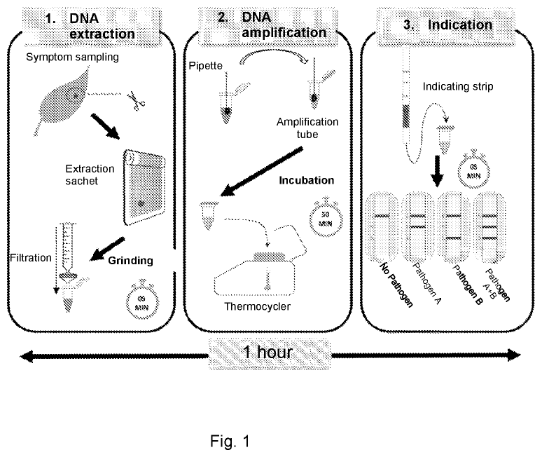

A synthetic representation of the steps of the method is shown in FIG. 1.

Amplification of specific sequences of the Zymoseptoria tritici pathogen in straw cereals such as soft wheat, barley and durum wheat is carried out by the PCR method. A person skilled in the art is familiar with this method. The specific elements developed in the context of the present disclosure are the primers and the interfering DNAs described below.

For amplification of the Zymoseptoria tritici pathogen:

Forward primer SEP_F1:
(SEQ ID NO. 1)
5′-[5′Digoxigenin]GCCTTCCTACCCCACCATGT-3′
Reverse primer SEP_R1: 5′-[5′6-FAM]
(SEQ ID NO. 2)
CCTGAATCGCGCATCGTTA-3′

For amplification of a mutation inducing resistance to MDR-type fungicides:

Non-specific MDR1 forward primer
for multi-resistance:
(SEQ ID NO. 3)
5′-[5′BiotinTEG]TACGAGACGAGAAAAAGACAG-3′
MDR2 reverse primer specific for
multi-resistance I:
(SEQ ID NO. 4)
5′-[5′ 6-FAM]GTCCTAATCGGGTAAAAGA-3′
MDR3 reverse primer specific for
multi-resistance II:
(SEQ ID NO. 5)
5′-[5′ 6-FAM]CCCCTAAGGTCAAAGAA-3′
MDR4 reverse primer specific for
multi-resistance III:
(SEQ ID NO. 6)
5′-[5′ 6-FAM]GGTAGGCTTGGCACTC-3′

For the addition of interfering DNA:

iDNA 1
(SEQ ID NO. 7)
5′-GGGTAGGA-3′
iDNA 2
(SEQ ID NO. 8)
5′-ACGATGCG-3′

The composition of the PCR mixture is detailed in Table 1.

TABLE 1
PCR mixture for detecting Zymoseptoria tritici
and MDR resistance in this pathogen

| Reagent | Volume |
|---|---|
| Taq Promega GoTaq | 5 μl |
| SEP_F | 0.25 μl to 5 μM |
| SEP_R | 0.25 μl to 5 μM |
| MDR1_F | 0.3 μl to 5 μM |
| MDR2_R | 0.3 μl to 5 μM |
| MDR3_R | 0.3 μl to 5 μM |
| MDR4_R | 0.3 μl to 5 μM |
| iDNA 1 | 0.4 μl at 20 μM |
| iDNA 2 | 0.4 μl at 20 μM |

The method is implemented by following the protocol below from a kit according to the present disclosure:
1—Collecting a leaf sample showing a symptom of infection
2—DNA extraction
3—Rehydrating the lyophilized pellet of the PCR mixture with rehydration buffer and adding the DNA extract of the sample
4—Amplifying sequences by PCR; for this step, a battery-operated JEULIN® machine (9 tubes) was used. It can be used close to the field

The PCR program used is as follows:

| | | | |
|---|---|---|---|
| 95° C. | 3 minutes | | |
| 95° C. | 3 seconds | | ×40 cycles |
| | | {close oversize brace} | |
| 60° C. | 20 seconds | | |

5—Indicating the result on a nitrocellulose strip to which antibodies are bound that are capable of recognizing the antigen markers located at the ends of the amplified primers by impregnating the strip with the mixture obtained

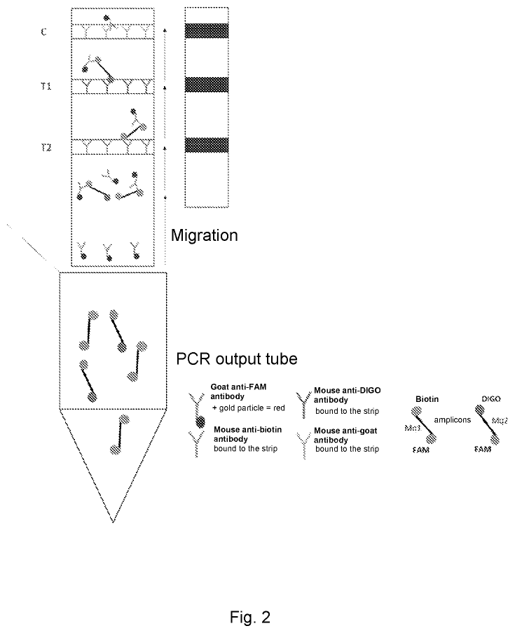

The principle of indicating the result on a strip is illustrated in FIG. 2.

### Example 2: Detection and Characterization of Strains of the Fusarium Pathogen with Indication of 4 Markers Using 2 Strips

This example illustrates the application of the method to the simultaneous detection of 4 markers to respond to a complex problem using a simple, rapid and inexpensive test that can be used near fields.

In practice, 2 strips are introduced into the same tube resulting from the same PCR reaction to reveal 4 markers; it is a quadruplex format. It can be used to respond to the following problem:
On wheat and corn, Fusarium are very frequent and cause very serious damage to the foliage, the roots, causing substantial lodging. Moreover, these Fusarium ssp. produce mycotoxins, some of which are lethal for humans and animals when their levels in food exceed certain limits (cumulative effects in tissues such as the liver, for example, nervous tissues, etc.);
These Fusarium produce Trichothecene-type mycotoxins (desoxynivalenol=DON, Nivalenol, T2 and HT2, Zearalenone, Fumonisin, etc.).

In the presence of Fusarium in a wheat field, it is possible to characterize the strains for their resistance to certain fungicides and simultaneously to know the type of mycotoxins they can produce. With 4 markers available, it is possible to:
use 2 markers for fungicide resistance (TSR and MDR or equivalent) and 2 markers for the most toxic types of mycotoxins, such as DON and Zearalenone, for example.
If the 2 fungicide resistance markers indicate that this Fusarium can effectively be eliminated, the harvest can be used for food even if the mycotoxin markers are positive;
If the two fungicide resistance markers indicate that this Fusarium cannot effectively be eliminated, the harvest can be used for food even if the mycotoxin markers are negative; however, it will be necessary to destroy the harvest if one of the mycotoxin markers is positive.

1. A field method for detecting the presence of a pest in a crop, comprising the steps of:
a. collecting a sample suspected of comprising a pest, the sample comprising a leaf of a plant suspected of being infected, or insect vectors of parasites, or weeds present in a crop;
b. extracting DNA or RNA from the sample;
c. using PCR to amplify the DNA or RNA sequences of the pest to determine the presence of the pest owing to a pair of specific primers making it possible to amplify a sequence characteristic of the pest; and
d. indicating the presence of the pest on an immunological strip;
wherein the amplification step is carried out in the presence of interfering DNA complementary to parts of the primers capable of forming polymers between them to avoid formation of aspecific bands on the immunological strips responsible for false positives.

2. The method of claim 1, further comprising detecting the presence of at least one other pest and/or determining at least one characteristic associated with a pest chosen from target-related pesticide resistance, multi-drug resistance, heat resistance, drought or humidity resistance.

3. The method of claim 1, further comprising simultaneously obtaining up to four results associated with detecting and/or characterizing one or more pests by using two strips in the same test.

4. The method of claim 1, wherein the pest to be detected is the fungus Zymoseptoria tritici, responsible for Septoria wherein the PCR amplification is carried out using a mixture comprising the following sequences:
a pair of specific primers making it possible to detect the presence of the fungus comprising sequences SEQ ID NO.1 and SEQ ID NO.2;
a pair of primers allowing the detection of multi-drug resistance comprising a primer of sequence SEQ ID NO.3 non-specific for multi-resistance and a primer chosen from:
a primer of sequence SEQ ID NO.4 specific for multi-resistance I;
a primer of sequence SEQ ID NO. 5 specific for multi-resistance II;
a primer of sequence SEQ ID NO.6 specific for multi-resistance III;
two interfering DNAs of sequences SEQ ID NO.7 and SEQ ID NO.8.

5. A kit for detecting the presence of a pest suitable for use in the field, comprising:
a lyophilized PCR reaction mixture comprising:
a pair of specific primers making it possible to amplify a sequence characteristic of the pest;
interfering DNAs complementary to the parts of the primers capable of forming polymers between them; and
a polymerase amplification enzyme; and
at least one immunological indicating strip making it possible to indicate the presence of the pest.

6. The kit of claim 5, wherein the PCR mixture further comprises at least one other pair of primers making it possible either to detect the presence of at least one other pest, or to determine at least one characteristic associated with a pest chosen from target-related pesticide resistance, multi-drug resistance, heat resistance, drought or humidity resistance.

7. The kit of claim 6, wherein the kit is configured to detect the presence of a pest and to characterize a pesticide resistance of this pest, wherein:
the PCR mixture further comprises one or more pairs of specific primers making it possible to amplify at least one mutation responsible for a pesticide resistance in the pest;
the strip comprises three distinct bands formed by the binding of antibodies specific for a particular antigen marker, namely respectively:
an antibody specific for an antigen marker located at the 5′ end of one of the primers allowing the amplification of a sequence specific for the pest;
an antibody specific for an antigen marker located at the 5′ end of one of the primers allowing the amplification of a sequence specific for a resistance; and
an antibody specific for an antigen marker common to all the pairs of primers making it possible to indicate the bands.

8. The kit of claim 5, comprising two immunological strips for simultaneously indicating four results from the same amplification, the results being chosen from detecting the presence of at least one pest and determining at least one characteristic associated with a pest chosen from target-related pesticide resistance, multi-drug resistance, heat resistance, drought or humidity resistance.

9. The kit of claim 5, wherein the kit is configured to detect multi-drug resistance of the fungus Zymoseptoria tritici, which is responsible for Septoria, wherein the lyophilized PCR mixture comprises:
the primers for detecting the presence of the pathogen are of sequence SEQ ID NO.1 and SEQ ID NO.2;
the primers allowing multi-resistance detection comprise a primer of sequence SEQ ID NO.3 non-specific for multi-resistance and a primer chosen from:
a primer of sequence SEQ ID NO.4 specific for multi-resistance I;
a primer of sequence SEQ ID NO.5 specific for multi-resistance II;
a primer of sequence SEQ ID NO.6 specific for multi-resistance III;
the interfering DNAs are of sequences SEQ ID NO.7 and SEQ ID NO.8.

10. A field method for detecting the presence of a pest in a crop or whether the pest presents a resistance of interest, using a kit according to claim 5, the method comprising the following steps:
A) collecting a sample suspected of comprising a pest, the sample comprising a leaf suspected of being infected, or insect vectors of parasites, or weed present in a crop;
B) extracting DNA or RNA from the sample;
C) introducing the extracted DNA or RNA into a tube containing the lyophilized PCR mixture;
D) performing PCR amplification; and
E) introducing one or two immunological strips for impregnation in the tube containing the amplicons to reveal whether the pest is present or whether the pest presents a resistance of interest.
